(19) United States
(12) Patent Application Publication
(10) Pub. No.: US 2025/0050034 A1
(43) Pub. Date: Feb. 13, 2025

(54) LIQUID MEDICATION ADMINISTRATION SYSTEM WITH FLOW REGULATING MODULE

(71) Applicant: NOVATIVE MEDICAL, LLC, West Jordan, UT (US)

(72) Inventor: Muhammed Usman Afzal, West Jordan, UT (US)

(21) Appl. No.: 18/792,385

(22) Filed: Aug. 1, 2024

Related U.S. Application Data
(60) Provisional application No. 63/531,092, filed on Aug. 7, 2023.

(51) Int. Cl. A61M 5/36; A61M 5/14; A61M 5/158; A61M 5/168; A61M 5/40; A61M 39/28

(52) U.S. Cl. CPC A61M 5/365; A61M 5/1413; A61M 5/158; A61M 5/16813; A61M 5/40; A61M 39/281; A61M 2005/1402; A61M 2205/3306

(57) ABSTRACT

A method of injecting primary liquid into a subject can include flowing primary liquid through a flow-regulating module with a pump. The flow-regulating module is configured to allow liquid flow and stop air flow. Liquid can be detected in the main line while the primary liquid flows through the flow-regulating module. Air can be detected in the main line after the primary liquid in the flow-regulating module, and then the flow valve of the flow-regulating module is closed when air is present in the main line. The pump can then cause the flowing of the clearing liquid into the main line behind with primary liquid without air entering the main line downstream from the flow-regulating module. As a result, the primary liquid is injected into the subject followed by clearing liquid without air.

### CROSS-REFERENCE TO RELATED APPLICATIONS

This patent application claims priority to U.S. Provisional Application No. 63/531,092 filed Aug. 7, 2023, which provisional is incorporated herein by specific reference in its entirety.

### BACKGROUND

### Field

The present disclosure relates to a system and method for providing liquid medication by injection into a subject with a sensor that activates a clearing liquid to follow the liquid medication.

### Description of Related Art

Previously, piggybacking has been used in intravenous drug administration to help utilize most or nearly all of the liquid medication. Piggybacking using a secondary line is a common practice in a clinical setting at the hospitals to administer various intravenous fluids, such as antibiotics, electrolytes, medications, and blood products. The secondary line is always used in conjunction with the primary line. A secondary line is comprised of a 250 ml saline bag, drip chamber, roller clamp, tubing, male-Luer, hanger, instruction-for-use, and packing material. However, piggybacking is problematic for a variety of reasons. Examples of piggybacking and the problems are included in the incorporated U.S. Provisional Application No. 63/531,092.

However, establishing a secondary line in the intravenous setup is expensive (e.g., material cost), and errors can occur. Often, secondary lines are difficult to set up, which causes errors that often require additional material or adds too much time to the procedure. Also, clinicians occasionally forget to open a roller-clamp on the secondary line that leads to a delay in therapy. Additionally, the entire medication in the tubing does not always get infused into the subject due to mismanagement and incorrect piggybacking. Moreover, it is commonly reported that there are frequent connections and disconnection of a secondary line from with the primary line, which has the potential to lead to blood stream infections. Thus, eliminating piggybacking from intravenous administrations can be advantageous.

### SUMMARY

In some embodiments, a drug administration system can include a flow-regulating module and a clearing liquid reservoir. The flow-regulating module can be adapted to be connected to a main line from a liquid medication reservoir. The flow-regulating module can include an air sensor, a flow valve, and a fluid output from the flow-regulating module. When the air sensor detects liquid the flow valve is open, and when the air sensor detects air the flow valve is closed. The clearing liquid reservoir can be fluidly coupled with fluid output from the flow-regulating module. The clearing liquid reservoir has a higher degree of resistance to fluid withdrawal, and the liquid medication reservoir has a lower degree of resistance to fluid withdrawal The fluid output from the flow-regulating module is configured to be fluidly coupled to a pump. As such, the pump preferentially pumps from the liquid medication reservoir due to the lower degree of resistance to fluid withdrawal compared to the clearing liquid reservoir's higher degree of resistance to fluid withdrawal. Once the liquid medication reservoir is empty, air enters the line to trip the flow-regulating module so that the main line is closed and clearing liquid from the clearing liquid reservoir begins to be withdrawn.

In some embodiments, the air sensor is configured to stimulate or optically probe liquid or air in the main line and differentiate between liquid and air.

In some embodiments of the drug administration system, the air sensor and flow valve include a float reservoir with a restrictor bottom and a float ball.

In some embodiments of the drug administration system, the flow valve includes a pinch valve.

In some embodiments of the drug administration system, the fluid output from the flow-regulating module includes a tube and/or an access port connector.

In some embodiments of the drug administration system, the main line and liquid medication reservoir are fluidly coupled with the flow-regulating module.

In some embodiments of the drug administration system, the pump is connected to the main line.

In some embodiments of the drug administration system, an injection needle is at a terminal position of the main line.

In some embodiments of the drug administration system, the clearing liquid reservoir is a syringe.

In some embodiments of the drug administration system, the clearing liquid reservoir includes a saline solution.

In some embodiments, a method of injecting a liquid medication into a subject is provided. The method can include providing the administration system of one of the embodiments. Liquid medication can be pumped through the flow-regulating module with the pump. Liquid is detected in the flow-regulating module. Then, air is detected after the liquid in the flow-regulating module. The system closes the flow valve of the flow-regulating module when air is detected. The clearing liquid is then pumped into the main line behind the liquid medication without air entering the main line downstream from the flow-regulating module. As a result, the liquid medication can be injected (e.g., intravenous administration) into the subject followed by clearing liquid without air.

In some embodiments, a method of injecting liquid medication into a subject is provided. The method can include flowing liquid medication through a flow-regulating module with a pump. The flow-regulating module is configured to allow liquid flow and stop air flow. Liquid can be detected in the main line while the liquid medication flows through the flow-regulating module. Air can be detected in the main line after the liquid medication in the flow-regulating module, and then the flow valve of the flow-regulating module is closed when air is present in the main line. The pump can then cause the flowing of the clearing liquid into the main line behind with liquid medication without air entering the main line downstream from the flow-regulating module. As a result, the liquid medication is injected into the subject followed by clearing liquid without air.

In some embodiments, the air is detected by an air sensor that is configured to stimulate or optically probe liquid or air in the main line and differentiate between liquid and air.

In some embodiments, the air is detected with an air sensor and the flow valve includes a float reservoir, a float ball, and a restrictor bottom.

In some embodiments, the method includes actuating the flow valve with an actuator.

In some embodiments, a float ball is caused to sink or fall when air is in the main line, when there is no liquid, the ball cannot float.

In some embodiments, the method can include allowing liquid medicine to flow through the main line and closing the main line once air is detected in the main line.

In some embodiments, the method can omit using a piggybacking setup.

In some embodiments, the clearing liquid reservoir is a syringe.

In some embodiments, the clearing liquid reservoir includes a saline solution.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A illustrates an embodiment of the intravenous drug administration system.

FIG. 1B illustrates another embodiment of the intravenous drug administration system.

FIG. 1C illustrates another embodiment of the intravenous drug administration system.

FIG. 2A illustrates an embodiment of a flow-regulating module.

FIG. 2B illustrates an embodiment of the flow-regulating module with the flow valve being a pinch valve.

FIG. 2C illustrates another embodiment of a flow valve having a first pincer and second pincer pinching the main line.

FIG. 3A illustrates another system that includes a flow-regulating module that has a shutoff valve containing a float reservoir with a restrictor bottom with a float ball therein.

FIG. 3B illustrates another system that includes a flow-regulating module that has a shutoff valve access port containing an access port and a float reservoir with a restrictor bottom with a float ball therein.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to a system that uses a flow-regulating module that has an air detector and a flow valve to control liquid medication administration with a clearing liquid provided to follow the liquid medication so that nearly all (preferably all) of the liquid medication is administered. The system omits the use of a secondary line piggybacked into the liquid medication line. Instead, the air detector detects air in the liquid medication line and implements the flow valve to stop flow in the liquid medication line, which then allows for the clearing liquid to flow into the liquid medication line to follow the liquid medication. As a result, minimal liquid medication remains in the line upstream from the connection with the clearing liquid. The pump can initially draw the liquid medication into the liquid medication line. When the air detector detects air in the liquid medication line behind the liquid medication, the module then causes the flow valve to stop flow in the liquid medication line upstream of the flow valve, downstream the pump operates to pull clearing liquid from a clearing liquid reservoir into the liquid medication line behind the liquid medication. This allows for substantially all of the liquid medication to be used, and only possible small amount between the flow valve and clearing liquid junction to be lost.

In some embodiments, the system includes an intravenous pump downstream of a flow-regulating module with an air detector and electromagnetic pinch valve as the flow valve. When the liquid medication is empty in the line and air is detected in the line, the pinch valve pinches the tubing and shuts off the flow from the liquid medication reservoir, which prevents any air from getting injected in the line downstream of the module from the liquid medication reservoir. Immediately after the main tubing is pinched, the clearing liquid (e.g., saline) is used as a flush that activates and starts to flush the line. The saline flush clears the liquid medication from the line and ensures that all the liquid medication in the tubing of the line is given to the subject receiving the treatment (e.g., patient). A port above the pump and syringe as the clearing liquid reservoir can be used for this setup.

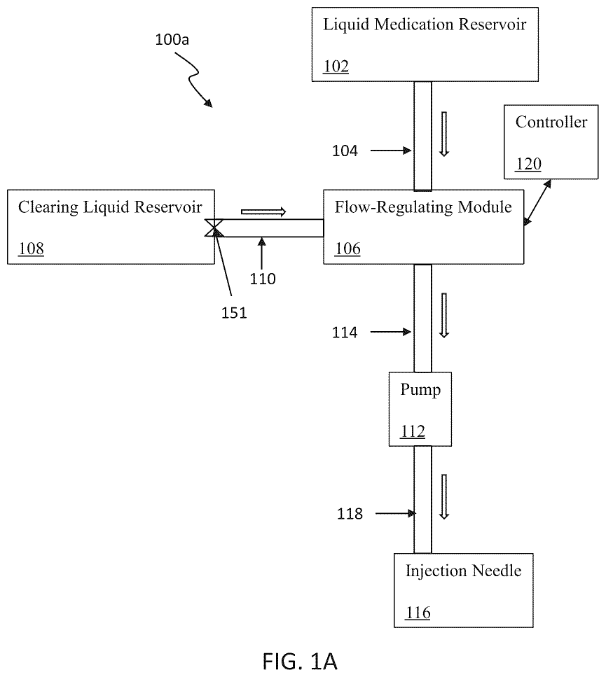

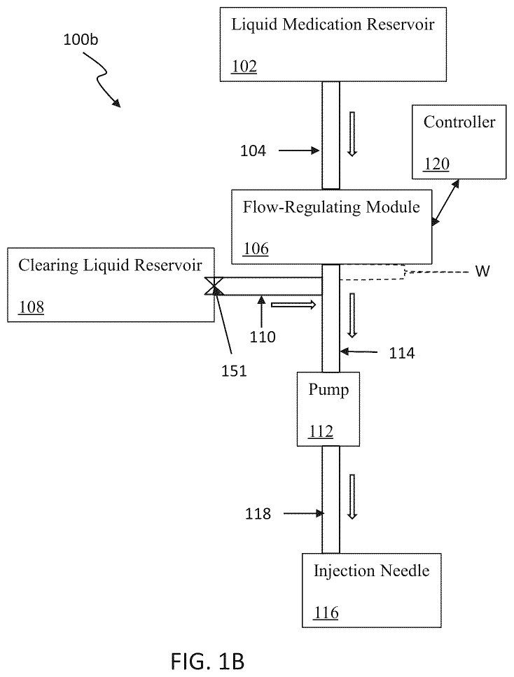

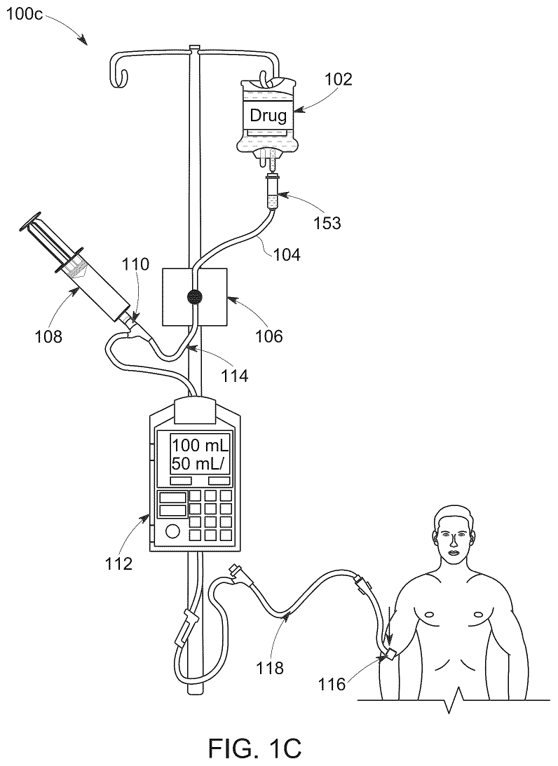

FIG. 1A illustrates an embodiment of the intravenous drug administration system 100a that includes the liquid medication reservoir 102 fluidly connected to the flow-regulating module 106 via main tubing 104. The flow-regulating module 106 is also fluidly connected to a clearing liquid reservoir 108 through a clearing fluid connector 110. The flow-regulating module 106 is fluidly connected to a pump 112 (e.g., intravenous pump) via main tubing 114. The main tubing 114 may be the same tube (e.g., downstream section) or a downstream tube from the main tubing 104. The pump 112 is fluidly connected to the injection needle 116 via tubing 118, which can be the same or different tube as the main tubing 104, 114. Nevertheless, the liquid medication reservoir is fluidly coupled with the injection needle 116 via the tubing by passing through the flow-regulating module 106 and pump 112. The liquid medication can be injected into the subject via the injection needle 116. The clearing liquid reservoir 108 is fluidly connected to the flow-regulating module 106 via clearing fluid connector 110, and there can be some junction, valve, combiner, or other feature at the intersection of clearing liquid supply into the liquid medication supply.

FIG. 1B illustrates an embodiment of the intravenous drug administration system 100b that includes the liquid medication reservoir 102 fluidly connected to the flow-regulating module 106 via main tubing 104. The flow-regulating module 106 is not fluidly connected to a clearing liquid reservoir 108. Instead, the clearing liquid reservoir 108 is connected to the tubing 114 (e.g., downstream of the flow-regulating module) by a distance W through the clearing fluid connector 110. The flow-regulating module 106 is fluidly connected to a pump 112 (e.g., intravenous pump) via main tubing 114. The clearing liquid reservoir is fluidly connected to the pump 112 via the secondary tubing and then the main tubing 114, via any type of junction or connector. The main tubing 114 may be the same tube (e.g., downstream section) or a downstream tube from the main tubing 104. The pump 112 is fluidly connected to the injection needle 116 via tubing 118, which can be the same or different tube as the main tubing 104, 114. Nevertheless, the liquid medication reservoir is fluidly coupled with the injection needle 116 via the tubing by passing through the flow-regulating module 106 and pump 112 before being injected into the subject via the injection needle 116. The clearing liquid reservoir 108 is fluidly connected to the main tubing 114 (e.g., downstream from flow-regulating module 106) via clearing fluid connector 110. There can be some junction, valve, combiner, or other feature at the intersection of clearing fluid connector 110 and the main tubing 114.

FIG. 1C illustrates an embodiment of the intravenous drug administration system 100c that includes the liquid medication reservoir 102 (e.g., medication bag) fluidly connected to the flow-regulating module 106 via main tubing 104. The clearing liquid reservoir 108 is connected to the tubing 114 (e.g., downstream of the flow-regulating module 106) by a distance W through the clearing fluid connector 110 (e.g., junction connector). The flow-regulating module 106 is fluidly connected to a pump 112 (e.g., intravenous pump) via main tubing 114. The clearing liquid reservoir 108 is fluidly connected to the pump 112 via the clearing fluid connector 110 and then the main tubing 114, via any type of fluidic junction or connector. The main tubing 114 may be the same tube (e.g., downstream section) or a downstream tube from the main tubing 104. The pump 112 is fluidly connected to the injection needle 116 via tubing 118, which can be the same or different tube as the main tubing 104, 114. Nevertheless, the liquid medication reservoir 102 is fluidly coupled with the injection needle 116 via the tubing by passing through the flow-regulating module 106 and the pump 112 before being injected into the subject via the injection needle 116. The clearing liquid reservoir 108 is fluidly connected to the main tubing 114 (e.g., downstream from flow-regulating module 106) via the clearing fluid connector 110.

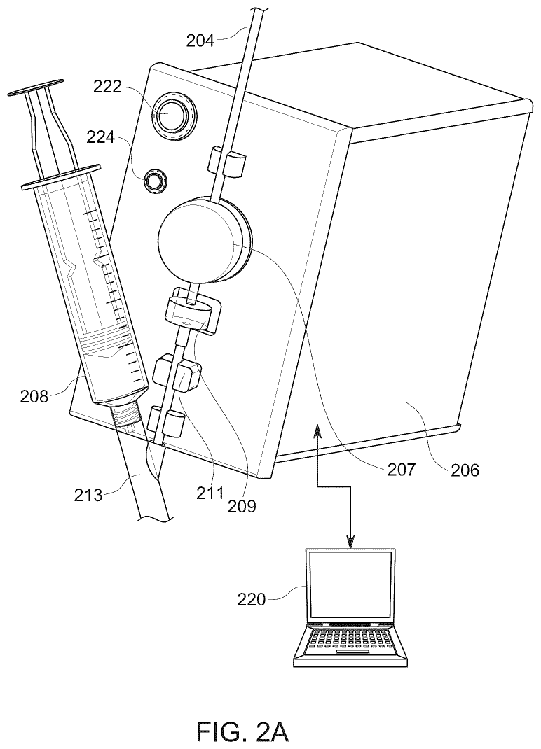

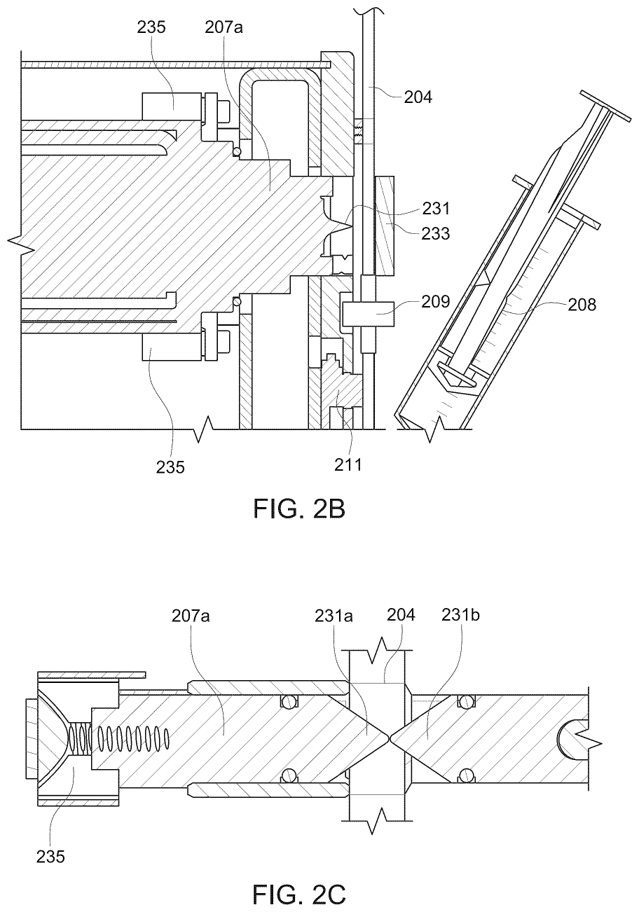

FIG. 2A illustrates an embodiment of a flow-regulating module 206. The flow-regulating module 206 is shown to include the main line 204 passing through a flow valve 207 and a check-valve 209, and past an air detector 211 before a Y-coupling 213 (e.g., access port coupling) with the clearing liquid reservoir 208 (e.g., syringe). A controller, (shown as controller 220), can be included in the flow-regulating module 206 to receive data from the air detector 211, and determine if there is liquid or air in the main line 204. The controller 220 can analyze the data and operate the flow valve 207 to allow flow when liquid and stop flow when air. Also, a power button 222 (or switch or other mechanism) is shown to turn the module 206 on and off. An activation button 224 (or switch or other mechanism) can activate the air detector 211 and pinch valve 207. Also, the controller 220 is shown to be communicatively coupled (e.g., wired, optical, wireless, etc.) with the module 206, so as to receive data from the air detector 211 and provide instructions to the flow valve 207 to open or close. Instructions and data may also be communicated between the controller 220 and pump.

FIG. 2B illustrates an embodiment of the flow-regulating module 206 with the flow valve 207 being a pinch valve 207a. The pinch valve 207a can include an actuated pincher 231 and pinch plate 233 that cooperate to squeeze the main line 204 to regulate to stop fluid flow in the tube of the main line 204. Actuators 235 can by hydraulic, mechanical, motor, linear motor, or other mechanism to drive the pincher 231 towards the pinch plate 233 to pinch the main line 204.

FIG. 2C illustrates another embodiment of a flow valve 207a having first pincer 231a and second pincer 231b pinching the main line 204.

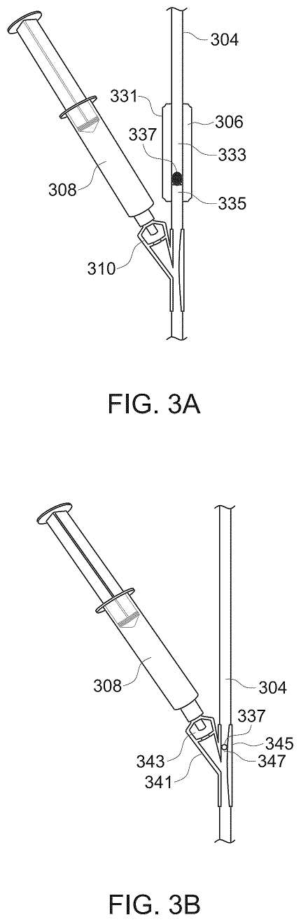

FIG. 3A illustrates another system that includes a flow-regulating module 306 that has a shutoff valve 331 containing a float reservoir 333 with a restrictor bottom 335 with a float ball 337 therein. When there is liquid flow within the main line 304 the float ball 337 floats within the float reservoir 333. When air enters into the float reservoir 333, the float ball 337 sinks or falls into the restrictor bottom 335 to seal and close the shutoff valve 331. The float ball 337 does not allow air to pass the restrictor bottom 335.

FIG. 3B illustrates another system that includes a flow-regulating module 306 that has a shutoff valve access port 341 containing an access port 343 and a float reservoir 345 with a restrictor bottom 347 with a float ball 337 therein. When there is liquid flow within the main line 304 the float ball 337 floats within the float reservoir 345. When air enters into the float reservoir 345, the float ball 337 sinks or falls into the restrictor bottom 347 to seal and close the shutoff valve 331. The float ball 337 does not allow air to pass the restrictor bottom 347. The shutoff valve access port 341 is shown to couple the clearing fluid reservoir 308 with the main line 304.

In some embodiments, the air detector can include an optical detector that can use optical means, such as refractive index, absorbance, inductance, reflectance, refraction, or other optical technique to distinguish between liquid and air. The air detector can include a sensor that has a light source, often an LED, and a light detector, such as a photodiode or phototransistor, housed in the module. The light source sends light to the detector through the tubing, or a cell connected to the tubing. The tubing or cell is usually made of a transparent material like plastic or glass. When air is in the line, the light emitted by the LED travels through the tube to the detector. When the liquid is in the line, the light is refracted out of the tube or cell due to the difference in the refractive indices of air and liquid. This means that when there is liquid in the main line, less or no light is passed to the detector or reflected back to the detector (depending on setup), so the flow is allowed. When light is detected, the flow is stopped by the controller. The sensor circuitry monitors the amount of light received by the detector. If the light level is low, the sensor determines that the liquid is in the line. If the light level increases past a threshold, it determines that there is air in the line.

In some embodiments, the sensor can be configured as a light sensor, ultrasonic sensor, capacitive sensor, conductivity sensor, pressure sensor, infrared sensor, float switch, or the like.

In some embodiments, the clearing liquid reservoir has stiction or has a stiction valve (e.g., 151 of FIG. 1A) associated therewith. Stiction is a term used to describe the static friction that must be overcome to initiate relative motion between two surfaces that are in contact and at rest. It is a combination of the words “static” and “friction.” Stiction is generally higher than the kinetic friction that occurs once the motion has started. As such, the syringe can provide stiction as there is a draw force from the pump to reach a certain level to draw from the clearing reservoir. Also, the clearing liquid reservoir has a higher degree of resistance to fluid withdrawal and the liquid medication reservoir has a lower degree of resistance to fluid withdrawal, comparatively. As such, the pump pulls liquid from the liquid medication reservoir before pulling from the clearing liquid reservoir.

During operation, the pump draws from the liquid medication reservoir that has less stiction than the clearing liquid reservoir. Comparatively, the liquid medication reservoir has less stiction than the clearing liquid reservoir or it has no stiction. Comparatively, the clearing liquid reservoir has more stiction or has stiction compared to the liquid medication reservoir. While a syringe is shown, other bladders, containers, or the like may require a draw force before the clearing liquid is drawn, whereas when there is liquid in the liquid medication reservoir no stiction allows the liquid medication to be preferentially drawn or drawn first.

In some embodiments, the drug administration system of any of the embodiments can be operated as described herein. The clearing liquid reservoir can include a 20 ml or 30 ml prefilled saline syringe that can be connected to the primary line access-port above the pump. Air can be purged from the access-port by pushing on the syringe plunger and inject a small volume of saline solution into the main line. The drip chamber (e.g., 153 of FIG. 1C) can be connected to the medication bag (e.g., liquid medication reservoir). The main line can be primed with the liquid medication. The tubing can be connected to the pump and flow-regulating module. The pump can be programmed pump the primary line and operated once started. Accordingly, the drug administration system is error proof with no delay in drug therapy. The entire bag of liquid medication can be used and administered to the subject without waste and without air getting in the line. There is significant reduction is risk for disconnections and risk for infection.

In some embodiments, the drug administration system can be used instead of a piggybacking intravenous setup. In some aspects, the flow-regulating module is used in conjunction with any intravenous pump. The flow-regulating module is equipped with an air detector and electromagnetic pinch valve. As the medication in the bag is emptied, air is detected in the line by an air detector, the pinch valve pinches the main tubing and shuts off the flow from the bag, which prevents any air to get injected in the line. Immediately after the main tubing is pinched, the saline flush clearing liquid activates automatically as the pump continues to run to deliver full volume of medication to the patient. This action flushes the line, which ensures that all the medication in the tubing is given to the patient. A 20- or 30-ml syringe can be used for this setup. Since the saline in the syringe does not flow freely as it would from a bag is ideal for this setup; thereby, eliminates the need for the secondary line setup.

The flow-regulating module can have different configurations such that the flow from the main line with the liquid medication can be completely occluded by different methods. Examples can include electromechanical devices equipped with an air-detection sensor (e.g., FIGS. 2A-2B). When air is detected in the line, the tubing can be pinched by several different mechanisms such as solenoid pinch-valve, servo motor, stepper motor, spring force, or the like (e.g., FIG. 2C). A ball-screw-servo-driven pinch tubing module can be designed to occlude the tubing. This flow-regulating module can be equipped with an air bubble detector.

In some embodiments, tubing is loaded into the module once it is primed with medication or other fluid to be injected into the subject (e.g., intravenous). A feature to hold the tubing can be used. Once the tubing is loaded in the module, the activate-button is pushed, which triggers the sensor to set to the fluid inside the tubing. When the fluid in the tubing runs out, the air is detected by the sensor, which triggers the servo motor to pinch the tubing. Once the tubing is fully occluded above the access-port, the flush syringe automatically activates as the pump continues to draw medication. A normal-saline (NS) syringe is selected in this method to flush the line due to its stiction property that it does allow NS to be drawn passively. In addition, it provides economic value to the user. Alternatively, a saline bag or any reservoir can potentially be utilized instead of a syringe.

Other methods to occlude the flow from the tubing can include a floating ball shutoff valve (e.g., as illustrated in FIGS. 3A-3B) that can be built-in to the access-port. When the liquid medication in the main tubing runs out, the floating ball lowers to the sealing surface (e.g., restrictor) and completely occludes the flow. Therefore, no air can be further introduced to the line. Additionally, these flow-regulating modules with the valve can be added to the infusion line (FIG. 3A) instead of being integrated to the access-port (FIG. 3B). The column that houses the flow ball stays in a vertical position during use, such as by handing or fixture that keeps in vertical position.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

While intravenous injections have been exemplified, the system and method can be configured for any type of injection, such as intramuscular, subcutaneous, intradermal, intra-articular, intrathecal, epidural, intracardiac, intraperitoneal, intraosseous, or other, as well as combinations thereof.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A drug administration system comprising:
a flow-regulating module adapted to be connected to a main line from a primary liquid reservoir, wherein the flow-regulating module includes an air sensor, a flow valve, and a fluid output from the flow-regulating module, wherein when the air sensor detects liquid the flow valve open, and when the air sensor detects air the flow valve is closed; and
a clearing liquid reservoir fluidly coupled with fluid output from the flow-regulating module, wherein the clearing liquid reservoir has a higher degree of resistance to fluid withdrawal and the primary liquid reservoir has a lower degree of resistance to fluid withdrawal,
wherein the fluid output from the flow-regulating module is configured to be fluidly coupled to a pump.

2. The drug administration system of claim 1, wherein the air sensor is configured to stimulate or optically probe liquid or air in the main line and differentiate between liquid and air.

3. The drug administration system of claim 1, wherein the air sensor and flow valve include a float reservoir, a float ball, and a restrictor bottom.

4. The drug administration system of claim 1, wherein the flow valve includes a pinch valve.

5. The drug administration system of claim 1, wherein the fluid output from the flow-regulating module includes a tube and/or an access port connector.

6. The drug administration system of claim 1, further comprising the main line and primary liquid reservoir fluidly coupled with the flow-regulating module.

7. The drug administration system of claim 6, further comprising the pump.

8. The drug administration system of claim 7, further comprising an injection needle.

9. The drug administration system of claim 1, wherein the clearing liquid reservoir is a syringe.

10. The drug administration system of claim 1, wherein the clearing liquid reservoir includes a saline solution.

11. A method of injecting a primary liquid into a subject, comprising:
providing the administration system of claim 1;
flowing primary liquid through the flow-regulating module with the pump;
detecting liquid in the flow-regulating module;
detecting air after the liquid in the flow-regulating module;
closing the flow valve of the flow-regulating module;
flowing clearing liquid into the main line behind with primary liquid without air entering the main line downstream from the flow-regulating module; and
injecting the primary liquid into the subject followed by clearing liquid without air.

12. A method of injecting primary liquid into a subject, comprising:
flowing primary liquid through a flow-regulating module with a pump, wherein the flow-regulating module is configured to allow liquid flow and stop air flow;
detecting liquid in the main line while the primary liquid flows through the flow-regulating module;
detecting air in the main line after the primary liquid in the flow-regulating module;
closing a flow valve of the flow-regulating module;
flowing clearing liquid into the main line behind with primary liquid without air entering the main line downstream from the flow-regulating module; and
injecting the primary liquid into the subject followed by clearing liquid without air.

13. The method of claim 12, wherein the air is detected by an air sensor that is configured to stimulate or optically probe liquid or air in the main line and differentiate between liquid and air.

14. The method of claim 12, wherein the air is detected with an air sensor and the flow valve includes a float reservoir, a float ball, and a restrictor bottom.

15. The method of claim 12, further comprising actuating the flow valve with an actuator.

16. The method of claim 12, further comprising allowing a float ball to sink or fall when air is in the main line.

17. The method of claim 12, further comprising allowing liquid medicine to flow through the main line and closing the main line once air is detected in the main line.

18. The method of claim 12, further comprising omitting a piggybacking setup.

19. The method of claim 12, wherein the clearing liquid is provided by a clearing liquid reservoir that is configured as a syringe.

20. The method of claim 19, wherein the clearing liquid includes a saline solution.
